(19) United States
(12) Patent Application Publication
(10) Pub. No.: US 2011/0166214 A1
(43) Pub. Date: Jul. 7, 2011

(54) METHODS AND COMPOSITIONS FOR DELIVERY OF TAXANES IN STABLE OIL-IN-WATER EMULSIONS

(75) Inventors: HARI R. DESU, NARASARAOPET (IN); KANAIYALAL R. PATEL, UNION, NJ (US); SATISH K. PEJAVER, BRIDGEWATER, NJ (US); NAVNEET PURI, BRIDGEWATER, NJ (US)

(73) Assignee: INNOPHARMA, LLC, PISCATAWAY, NJ (US)

(21) Appl. No.: 12/986,909

(22) Filed: Jan. 7, 2011

Related U.S. Application Data
(60) Provisional application No. 61/293,195, filed on Jan. 7, 2010.

(51) Int. Cl. A61K 31/337; A61K 8/02; A61P 35/04

(52) U.S. Cl. 514/449; 514/786; 514/773; 514/772.5; 514/784

(57) ABSTRACT

The present invention provides methods and compositions for delivery of taxanes in stable oil-in-water emulsion. The inventive emulsion formulation includes an oil phase, aqueous and emulsifier phases. The oil portion includes all or substantial amount of taxane, vegetable oil and medium chain triglycerides; aqueous phase includes an emulsion stabilizer; emulsifier phase reduces the surface tension between oil and aqueous phases to produce a stable oil-in-water emulsion. The inventive compositions produce minimal side effects upon administration.

### CROSS-REFERENCE TO RELATED APPLICATIONS

This patent application claims the benefit of U.S. Provisional Patent Application No. 61/293,195, filed Jan. 7, 2010, which is incorporated by reference.

### FIELD OF INVENTION

The present invention relates to pharmaceutical compositions containing taxanes. Specifically, the present invention relates to stable oil-in-water emulsions of taxanes.

### BACKGROUND OF THE INVENTION

Docetaxel is a potent anticancer agent belonging to the taxane family. Docetaxel is prepared by a semi-synthetic production method beginning with a precursor extracted from the renewable needle biomass of yew plants. Docetaxel is used for treating patients with breast and non-small cell lung cancer. Due to their toxic nature, taxoid drugs, such as docetaxel, are almost always administered via injection or infusion of liquid solutions. Because of its poor solubility in water, the current commercial formulation of docetaxel poses challenges for pharmaceutical scientists and physicians as well as serious side effects for the patient.

The current state of the art docetaxel formulation (TAXOTERE™) requires high concentrations of organic solvents and toxic surfactants in order to solubilize the drug. Unfortunately, the TAXOTERE™ formulation leads to high rates of hypersensitivity reactions upon intravenous administration. These hypersensitivity reactions have been attributed to the high concentration of polysorbate 80 required to solubilize and stabilize the formulation. As a result, the FDA has mandated that the manufacturers of TAXOTERE™ include a “black box” warning label in the prescribing information about hypersensitivity reactions. Accordingly, TAXOTERE™ must not be given to patients who have a history of severe hypersensitivity reactions to TAXOTERE™ or to other drugs formulated with polysorbate 80.

Numerous lipid based drug delivery approaches including lipid capsules, liposomes and emulsions in injectable formulations have been pursued—the most successful of which have been the incorporation of taxane drugs into emulsion and liposome based formulations. U.S. Patent Application Publication No. 2005/0214378 describes a composition of taxane in lipid solution. However, no mention is made if the proposed composition is stable enough for encapsulation of high amounts of taxane drugs. International Patent Application Publication WO 2009/062398 discloses a liposome approach to deliver taxane drugs. The liposome formulation significantly loses docetaxel encapsulation over 3 months of storage and also is difficult for high drug incorporation into the liposomes.

Similarly, attempts to formulate docetaxel into a stable lipid emulsion have been unsuccessful. U.S. Pat. No. 6,458,373 describes an oil-in-water emulsion consisting of vitamin E as a carrier oil in which taxane is dissolved together with surfactants. However, the emulsion failed to show efficacy compared to Taxol™ and the emulsion formulation exhibited severe toxic effects. Additionally, oil-in-water emulsions incorporating docetaxel have been formulated with Tributyrin as the oil phase. However, such emulsions have demonstrated high toxicity. See, e.g., J. C. Leroux et al., “An Investigation on the use of Tributyrin Nanoemulsions for Docetaxel Delivery”, J. Drug Del. Sci & Technol. 18, pp 189-195 (2008).

Therefore, there is a need for a stable and efficacious taxane formulation with reduced side effects.

### SUMMARY OF THE INVENTION

The present invention generally relates to pharmaceutical compositions containing a taxane. More specifically, the pharmaceutical compositions comprise a taxane, an oil phase, an aqueous phase, and an emulsifier. The inventive compositions are stable and less toxic and contain therapeutic concentrations of taxanes.

The present invention is directed to a pharmaceutical composition comprising:
(a) a taxane;
(b) an oil phase, wherein the oil phase is present in an amount of at least about 4.0% w/w of the total composition;
(c) an aqueous phase; and
(d) an emulsifier, wherein the emulsifier is present in an amount less than about 1.2% w/w or more than 5.0% w/w of the total composition.

The invention further relates to a pharmaceutical composition comprising:
(a) docetaxel;
(b) an oil phase comprising vegetable oil and medium chain triglycerides, wherein the oil phase is present in an amount of at least about 4.0% w/w of the total composition, wherein the vegetable oil is present in an amount of at least about 3.0% w/w of the total composition, and/or wherein the medium chain triglycerides are present in an amount of about 3.0% w/w of the total composition;
(c) an aqueous phase selected from the group consisting of an aqueous solution of polyvinylpyrrolidone and/or an aqueous solution of human serum albumin, wherein the aqueous phase is present in an amount of about 0.05% to about 50.0% w/w of the total composition; and
(d) an emulsifier comprising phospholipids, wherein the emulsifier is present in an amount less than about 1.2% w/w or more than 5.0% w/w of the total composition.

In one embodiment, the emulsifier can be lecithin, wherein the lecithin is present in an amount of less than about 1.2% w/w (e.g., 1.0% w/w or less) of the total composition. In another embodiment, the emulsifier can be cardiolipin, cholesterol, or sodium cholesterol sulfate present in an amount of less than about 1.0% w/w of the total composition.

The present invention also is directed to an oil-in-water emulsion comprising:
(a) docetaxel in an amount of about 0.01% to about 10.0% w/w of the total emulsion;
(b) an oil phase comprising vegetable oil and medium chain triglycerides and, wherein the oil phase is present in an amount of at least about 4.0% w/w of the total emulsion, wherein the vegetable oil is present in an amount of at least about 3.0% w/w of the total emulsion, and wherein the medium chain triglycerides are present in an amount of about 3.0% w/w of the total emulsion;
(c) an aqueous phase selected from the group consisting of an aqueous solution of polyvinylpyrrolidone and/or an aqueous solution of human serum albumin, wherein the aqueous phase is present in an amount of about 0.05% to about 50.0% w/w of the total emulsion; and
(d) an emulsifier comprising phospholipids, wherein the emulsifier is present in an amount less than about 1.2% w/w or more than 5.0% w/w of the total emulsion.

In one embodiment, the emulsifier can be lecithin, wherein the lecithin is present in an amount of less than about 1.2% w/w (e.g., 1.0% w/w or less) of the emulsion. In another embodiment, the emulsifier can be cardiolipin, cholesterol, or sodium cholesterol sulfate is present in an amount less than about 1.0% w/w of the total composition.

The inventive formulation preferably contains stabilizing agents, buffer components, anti-oxidants, isotonicity adjusting agents and lyoprotective agents. The pharmaceutical compositions are stable for months at room temperature. The present inventive compositions can be subjected to sterile filtration with ease. The inventive compositions may be in liquid or lyophilized forms. The lyophilized compositions can be reconstituted with intravenous diluents such as saline, dextrose, or water for injection without crystallization of taxane. Other advantages of the present invention include multiple presentations of the inventive compositions, such as in ampoules, vials, prefilled syringes, or intravenous bags.

### BRIEF DESCRIPTION OF THE SEVERAL VIEWS OF THE DRAWING(S)

FIGS. 1A and 1B are graphs demonstrating the particle size distribution of the inventive docetaxel emulsion as determined by the photon correlation spectroscopy particle sizing method (Delsa™ Nano analyzer; Beckman Coulter). FIG. 1A demonstrates the intensity distribution with percent intensity (differential and cumulative) on the y-axes and diameter (nm) on the x-axis. FIG. 1B demonstrates ACF with G2(T) on the y-axis and time (μ seconds) on the x-axis.

FIGS. 2A and 2B are graphs demonstrating the zeta potential of the inventive docetaxel emulsion as determined by the electrophoretic light scattering method. FIG. 2A is an EOS plot with frequency (Hertz) and zeta potential (mV) on the x-axes. FIG. 2B demonstrates mobility distribution with intensity on the y-axis and frequency (Hertz) and zeta potential (mV) on the x-axes.

### DETAILED DESCRIPTION OF THE INVENTION

The present invention is related to providing a pharmaceutical formulation of taxanes in a liquid dispersed dosage form including at least a lipid fraction in addition to the taxane, wherein the composition is stable in aqueous dispersion at room temperature for at least 24 hours. Preferably, the dispersed dosage form is an oil-in-water emulsion containing oil, water, and emulsifiers. Preferably, the taxane is either docetaxel or paclitaxel. More preferably, the taxane is docetaxel. Preferably, the composition is free or substantially free of taxane drug crystals and, in the preferred embodiment, is free of docetaxel crystals or precipitate in the formulation.

As used herein, taxanes refer to a class of anticancer compounds widely used in cancer chemotherapy. Alternative terms for taxanes include taxines. Preferred taxane compounds for use in the inventive formulation include docetaxel and paclitaxel. More preferably, the taxane compound is docetaxel. Other taxanes include 10-oxo-docetaxel, epi-docetaxel; 7-epi-10-oxo-docetaxel; 6-hydroxy taxol; 7-epipaclitaxel; t-acetyl paclitaxel; 10-desacetyl-paclitaxel; 10-desacetyl-7-epipaclitaxel; 7-xylosylpaclitaxel; 10-desacetyl-7-glutarylpaclitaxel; 7-L-alanylpaclitaxel; spicatin; cephalomannine; cephalomannine-7-xyloside; 7-epi-10-deacetylcephalomannine; 10-deacetylcephalomannine; taxayuntin; N-debenzoyltaxol A; O-acetylbaccatin IV; 7-(triethylsilyl)baccatin III; baccatin III 13-O-acetate; baccatin diacetate; baccatin; baccatin VII; baccatin VI; baccatin IV; 7-epi-baccatin III; baccatin V; baccatin I; baccatin III; and baccatin A

The taxane can be present in the pharmaceutical composition in any suitable amount. The taxane is generally present in a therapeutically effective amount to treat a condition in a human patient. In a preferred embodiment, the taxane is present in a therapeutically effective amount to treat cancer. Typically, the taxane is present in an amount of about 0.01% to about 10.0% w/w of the total composition (e.g., about 0.05%, about 0.1%, about 0.5%, about 1.0%, about 2.0%, about 3.0%, about 4.0%, about 5.0%, about 6.0%, about 7.0%, about 8.0%, or about 9%).

The present invention provides oil-in-water emulsion formulation of a taxane drug, wherein the formulation comprises one or more components that are able to form emulsions. For example, the formulation can comprise oil, aqueous, and emulsifier phases.

Preferably, the oil phase of the formulation comprises vegetable oil and/or medium chain triglycerides derived from natural, semi-synthetic or synthetic origin.

The oil phase in the inventive composition can comprise any suitable oils derived from hydrocarbons or carbohydrates that are liquids at 37° C. Oils include glycerides or non-glycerides. Preferably, the oil phase of the formulation comprises vegetable oil, medium chain triglycerides, or mixtures thereof.

Glycerides can be a mono-, di-, tri-glycerides, or a mixture thereof. In certain embodiments, the oil refers to a vegetable oil derived from plant seeds or nuts. Vegetable oils are typically “long-chain triglycerides,” formed when three fatty acids (usually about 14 to about 22 carbons in length) form ester bonds with the three hydroxyl groups on glycerol. In certain embodiments, vegetable oils may be hydrogenated to increase the loading efficiency of drugs inside the oil phase. Examples include, but are not limited to, soybean oil, safflower oil, sesame oil, corn oil, almond oil, canola oil, palm kernel oil, coconut oil, linseed oil, peanut oil, rapeseed oil, and the like. Preferably, the vegetable oil is a refined soybean oil or safflower oil.

Another suitable component of the oil phase is medium chain triglycerides (MCTs). MCTs are triglycerides derived from natural, semi-synthetic or synthetic origin. MCTs are made from fatty acids that are usually about 8 to about 12 carbons in length. In other words, suitable MCTs generally have an aliphatic carbon chain length between C8 to C12. MCTs have been used in emulsions intended for total parenteral nutrition.

Suitable MCTS includes caproic, caprylic, capric and lauric triglycerides, and mixtures thereof. A preferred MCT is Miglyol® produced by SASOL Germany GmbH which are distillation fractions of coconut oil. More preferably, the Miglyol is Miglyol® 812 that is a caprylic/capric triglyceride that contains 55% C₈ and 45% C₁₀ fatty acids. Most preferably, the Miglyol® is Miglyol® 812N.

The oil phase is present in the pharmaceutical composition in an amount of at least about 4.0% w/w of the total composition (e.g., at least about 5.0%, at least about 7.0%, at least about 10.0%, at least about 12.0%, at least about 15.0%, at least about 17.0%, at least about 20.0%, or at least about 25.0%). When vegetable oil is included in the oil phase, it is preferably present in an amount of at least about 3.0% w/w of the total composition. When medium chain triglycerides are included in the oil phase, it is preferably present in an amount of at least about 3.0% w/w of the total composition. Moreover, the ratio of oil phase to taxane drug used in the inventive formulation typically is at least about 8:1 by weight ratio, and more preferably at least about 10:1 by weight ratio. Typically, the ratio of oil phase to taxane drug used in the inventive formulation is at least about 12:1 by weight ratio, such as at least about 16:1 or about 20:1 by weight ratio. However, the ratio of oil phase to taxane used in the inventive formulation does not exceed about 50:1 by weight ratio. Preferably, the ratio of oil phase to taxane agent is between 8:1 and 50:1 by weight ratio.

The aqueous phase of the pharmaceutical composition can comprise any suitable component or mixture of components. The components in the aqueous phase are compatible with water. In one preferred embodiment, the aqueous phase comprises polyvinylpyrrolidone. Polyvinylpyrrolidone can be present in an amount of about 0.05% to about 50.0% w/w of the total composition (e.g., about 0.1%, about 0.5%, about 1.0%, about 5.0%, about 10.0%, about 15.0%, about 20.0%, about 25.0%, about 30.0%, about 35.0%, about 40.0%, or about 45.0%). In another preferred embodiment, the aqueous phase comprises human serum albumin. Human serum albumin can be present in an amount of about 0.05% to about 50.0% w/w of the total composition (e.g., about 0.1%, about 0.5%, about 1.0%, about 5.0%, about 10.0%, about 15.0%, about 20.0%, about 25.0%, about 30.0%, about 35.0%, about 40.0%, or about 45.0%).

The inventive compositions can further comprise one or more water-soluble components. A preferred water-soluble component is polyvinylpyrrolidone homopolymers, which also is referred to as polyvinylpyrrolidone. The polyvinylpyrrolidone homopolymers reduce coalescence of oil droplets and improve emulsion stability. Preferably, the molecular weight of the polyvinylpyrrolidone homopolymer is in the range of 5,000 to 2,000,000 Da. More preferably, the molecular weight of the polyvinylpyrrolidone homopolymer is in the range of 8,000 to 1,500,000 Da. The polyvinylpyrrolidone homopolymer preferably is supplied in a powder form and has a glass transition temperature in the range of 100° C. to 200° C.

In another embodiment, the present invention can comprise an emulsion stabilizing protein. In certain embodiments of the present invention, the emulsion stabilizing protein can include, but is not limited to, albumins, immunoglobulins, caseins, hemoglobins, lysozymes, alpha.-2-macroglobulin, fibronectins, vitronectins, fibrinogens and lipases. Proteins, peptides, enzymes, antibodies and combinations thereof, are general classes of stabilizers contemplated for use in the present invention. Preferably, the protein for use in the inventive formulation is human serum albumin.

The pharmaceutical compositions of the present invention contain an emulsifier. Emulsifiers are surface active molecules that adsorb to the surface of oil droplets during homogenization, forming a protective membrane that prevents the droplets to aggregate. Emulsifiers contain both polar and non-polar regions. Emulsifiers for use in the compositions of the present invention can include, but are not limited to, lipids, proteins, small molecule surfactants, monoglycerides and sucrose esters of fatty acids. Emulsifiers used in the inventive formulations can be derived from natural, semi-synthetic or synthetic origin.

The emulsifier present in the present invention can comprise any suitable emulsifier that is compatible with the oil phase and aqueous phase. The emulsifier can be a lipid or at least two lipids. Preferably, the emulsifier is a phospholipid, a lipid derivative, a lipid salt and mixtures thereof. More preferably, the pharmaceutical composition contains at least two phospholipids. Most preferably, in pharmaceutical compositions containing at least two phospholipids, one of the phospholipids is egg lecithin and another of the phospholipids is cardiolipin or cardiolipin derivative. Cardiolipin is also referred to as 1,3-bis(sn-3′-phsphatidyl)-sn-glycerol. Alternatively, the pharmaceutical composition contains at least one phospholipid and at least one cholesterol or cholesterol derivative.

Suitable phospholipids include unsaturated phospholipids and saturated phospholipids. Suitable unsaturated phospholipids can include, for example, 1,2-dimyristoleoyl-sn-glycero-3-phosphatidylcholine, 1,2-dipalmitoleoyl-sn-glycero-3-phosphatidylcholine, 1,2-dioleoyl-sn-glycero-3-phosphatidylcholine (DOPC), 1,2-dipalmitoleoyl-sn-glycero-3-phosphatidylethanolamine, 1,2-dioleoyl-sn-glycero-3-phosphatidylethanolamine (DOPE), 1,2-dioleoyl-sn-glycero-3-phosphatidylglycerol, 1,2-dioleoyl-sn-glycero-3-phosphatidylserine (DOPS), 1,2-dioleoyl-sn-glycero-3-phospho-(1′-myo-inositol-3′,4′-biphosphate) and 1′,3′-bis(1,2-dimyristoyl-sn-glycero-3-phospho)-sn-glycerol. Suitable saturated phospholipids can include, for example, dimyristoyl phosphatidylcholine (DMPC), distearoylphasphatidylcholine (DSPC), dipalmitoylphosphatidylcholine (DPPC), dimyristoyl phosphatidylethanolamine (DMPE), distearoylphasphatidylethanolamine (DSPE), dipalmitoylphosphatidylethanolamine (DPPE), dimyristoyl phosphatidylserine (DMPS), distearoylphasphatidylserine (DSPS), dipalmitoylphosphatidylserine (DPPS), dimyristoyl phosphatidylglycerol (DMPG), distearoylphasphatidylglycerol (DSPG), dipalmitoylphosphatidylglycerol (DPPG), hydrogenated purified soybean phosphatidylcholine (HSPC), hydrogenated purified egg yolk, phosphatidylcholine and 1′,3′-bis[1,2-dimyristoyl-sn-glycero-3-phospho]-sn-glycerol.

Another suitable phospholipid is lecithin. Lecithin is an example of a mixture of phospholipids containing phosphatidylcholine, phosphatidylethanolamine, phosphatidylinositol and free fatty acids. Preferably, lecithin is egg-derived lecithin, known as egg lecithin. Saturated lipids useful in the present invention include cholesterol or sodium cholesterol sulfate.

The emulsifier is present in the pharmaceutical composition in an amount of less than about 1.2% w/w or more than about 5.0% w/w. Preferably, the emulsifier is egg lecithin in an amount of less than about 1.2% w/w or more than about 5.0% w/w. More preferably, the emulsifier is egg lecithin in an amount of about 1.0% w/w or about 5.1%. In another preferred embodiment, the emulsifier is egg lecithin and cardiolipin in an amount of less than about 1.2% w/w or more than about 5.0% w/w. More preferably, the emulsifier is egg lecithin and cardiolipin in an amount of about 1.1%

In one embodiment, the emulsifier is present in an amount of less than about 1.2% w/w (e.g., less than or equal to about 1.0%, less or equal to about 0.8%, less or equal to about 0.5%, less or equal to about 0.2%, or less or equal to about 0.1%) of the total composition. In this embodiment, the composition preferably further comprises a polyvinylpyrrolidone homopolymer or emulsion stabilizing protein. More preferably, the polyvinylpyrrolidone homopolymer is polyvinylpyrrolidone and the emulsion stabilizing protein is human serum albumin. Polyvinylpyrrolidone and human serum albumin can be present in any suitable amount typically about 0.05% to about 50.0% w/w of the total composition.

In this embodiment, when lecithin is included as an emulsifier, it is preferably present in an amount of about 1.0% w/w or less (e.g., about 0.8%, about 0.5%, about 0.2%, or about 0.1%) of the total composition. When present, cardiolipin, cholesterol, or sodium cholesterol sulfate is present in an amount less than about 1.0% w/w (e.g., about 0.8%, about 0.5%, about 0.2%, or about 0.1%) of the total composition. When lecithin and cardiolipin are both included as emulsifiers, they are present in a combined amount of about 1.2% w/w or less (e.g., about 1.1%, about 1.0%, about 0.8%, about 0.5%, about 0.2%, or about 0.1%).

In another embodiment, the emulsifier is present in an amount of more than about 5.0% w/w (e.g., more than or equal to about 5.1%, more than or equal to about 5.5%, more than or equal to about 6.0%, more than or equal to about 7.0%, more than or equal to about 8.0%, more than or equal to about 9.0%, more than or equal to about 10.0%, more than or equal to 15.0%, or more than or equal to 20.0%) of the total composition. In this embodiment, the composition preferably does not comprise a polyvinylpyrrolidone homopolymer or emulsion stabilizing protein (e.g., polyvinylpyrrolidone or human serum albumin).

In this embodiment, when egg lecithin is included as an emulsifier, it is preferably present in an amount of about 5.0% w/w or more (e.g., about 5.1%, about 5.5%, about 6.0%, about 7.0%, about 8.0%, about 9.0%, about 10.0%, about 15.0%, or about 20.0%) of the total composition. When egg lecithin and cardiolipin are both included as emulsifiers, they are present in a combined amount of about 5.0% w/w or more (e.g., about 5.1%, about 5.5%, about 6.0%, about 7.0%, about 8.0%, about 9.0%, about 10.0%, about 15.0%, or about 20.0%).

The pharmaceutical compositions of the present invention optionally can include other pharmaceutically acceptable excipients, such as, for example, buffers, preservatives, antioxidants, isotonicity agents, lyoprotectants, and mixtures thereof.

In the present invention, the pH of the emulsion may vary. In certain embodiments, the pH of the emulsions is between 3 and 7 (e.g., 4, 5, or 6). Suitable buffers used in the inventive formulations may include citric, acetic, maleic, phosphoric, succinic, or tartaric acid, and the counter ion salts thereof. In certain embodiments, the molar concentration of the buffer is between 5 mM to 150 mM (e.g., 10 mM, 20 mM, 30 mM, 40 mM, 50 mM, 60 mM, 70 mM, 80 mM, 90 mM, 100 mM, 110 mM, 120 mM, 130 mM, or 140 mM).

To further improve the stability, the present formulation typically includes one or more anti-oxidants. Lipid soluble anti-oxidants such as, butylated hydroxytoluene, butylated hydroxyanisole, propyl gallate, and α-tocopherol, or water soluble anti-oxidants, such as sodium EDTA and thioglycerol may be included in the inventive compositions. In certain embodiments, the anti-oxidant concentration is between 0.005% and 5% w/w of the total composition (e.g., 0.01%, 0.05%, 0.1%, 0.5%, 1.0%, 2.0%, 3.0%, or 4.0%).

The emulsions of the present invention can further comprise one or more lyoprotectants to enhance the stability of the formulation during lyophilization. Any suitable lyoprotectants including sugars, amino acids, and polymers may be included in the inventive compositions. Preferably, sugars such as mannitol, sucrose, and trehalose; amino acids such as lysine and alanine; polymers, such as proteins and polyvinylpyrrolidone, can be included in the present formulations.

Typically, the lyoprotectant represents less than 50.0% w/w of the total composition. Where the lyoprotectant represents as little as about 1.0% w/w of the total composition, it can enhance the stability of the proposed formulations. More typically, the lyoprotectant represents at least about 5.0% w/w or at least about 10.0% w/w or at least about 20.0% w/w of the total composition. The inventive compositions further can comprise one or more isotonicity agents, which preferably is sodium chloride or glycerol or thioglycerol or a sugar.

The emulsions of the present invention preferably are formed by steps that include low shear homogenization of the emulsions between 5000 and 25,000 revolutions per minute (RPM). More preferably, low shear homogenization is performed between 9,500 and 25,000 RPM. The mixture formed by low shear homogenization is further subjected to high shear homogenization or microfluidization at 18,000 to 50,000 PSI, and more preferably at 22,000 to 30,000 PSI. The pharmaceutical compositions of the invention may be in the form of an emulsion, preferably an oil-in-water emulsion. In one embodiment, the oil-in-water emulsion may have oil phase droplets having mean diameters of about 100 nm to about 1000 nm dispersed in the aqueous phase.

In certain embodiments, the invention emulsion formulation can be filtered through 0.22 micron filters. In certain embodiments, the proposed formulation may be subjected to heat sterilization to render the emulsions as sterile liquid formulations.

In an alternative embodiment, the pharmaceutical compositions of the invention can be in the form of a lyophilized powder. Suitable lyophilization techniques known to those skilled in the art may be used. In one embodiment, the oil-in-water emulsion of the present invention is subject to lyophilization to obtain a powder. In the present invention, one or more lyoprotectants can be included to retain the droplet mean diameter of the emulsions as before lyophilization.

The inventive emulsions or lyophilized powders of the invention can be diluted or reconstituted with standard intravenous diluents known to those in the art. Suitable intravenous diluents for use in the present invention include water, saline, dextrose 5% in water, water for injection or lactated ringer's solution. Upon dilution or reconstitution of the lyophilized composition, the inventive composition may still be an oil-in-water emulsion ready for intravenous infusion. Preferably, dilution or reconstitution yields the oil-in-water emulsion of the present invention, and the emulsion retains a similar droplet mean diameter as before lyophilization.

Upon dilution of the inventive emulsions or reconstitution of the lyophilized powders of the present invention with intravenous diluents, an intravenous infusion is formed whose total volume may not exceed 300 mL to yield a taxane concentration of approximately 1 mg/mL. Preferably, the taxane of the inventive composition is docetaxel.

The inventive emulsion can be presented as an ampoule, vial, pre-filled syringe device, or intravenous bag. In a similar manner, the lyophilization powder of the present invention can be presented in a vial or pre-filled syringe device.

The pharmaceutical compositions of the invention can be used in the treatment of cancer in a human patient. The pharmaceutical compositions may be diluted for administration with standard parenteral dilution solutions known to those of skill in the art. The dosage of the composition to be administered can be any therapeutically effective dosage as determined by those of skill in the art. The diluted or reconstituted intravenous infusions of the present inventive compositions are administered to treat cancer. The cancer that may be treated includes, for example, breast cancer, lung cancer, colon cancer, prostate cancer, stomach cancer, head-and-neck cancer or ovarian cancer.

The following examples further illustrate the invention but, of course, should not be construed as in any way limiting its scope.

### Example 1

This example demonstrates the preparation of a stable oil-in-water emulsion of the invention.

An emulsion was prepared as described in Table 1.1 using polyvinylpyrrolidone (PVP 17 PF) polymer as a stabilizer.

TABLE 1.1
Composition of PVP 17 PF Stabilized Docetaxel Emulsion

| Ingredient | Percentage (% w/w) |
|---|---|
| Docetaxel | 0.5 |
| Soybean oil | 3.1 |
| Miglyol 812N | 3.1 |
| alpha-tocopherol | 4.3 |
| Egg lecithin | 1.0 |
| Cardiolipin | 0.1 |
| 2% PVP 17 PF solution | q.s. 100 |

A solution of 0.5 g/35 mL docetaxel was dissolved in ethanol. Soybean oil, Miglyol 812N and α-tocopherol were added to the ethanol solution with continuous stirring to form a solution. Ethanolic solution of egg lecithin and methylene chloride solution of cardiolipin were added to the oil phase. The docetaxel solution was subjected to vacuum evaporation for removal of ethanol and methylene chloride. A 2% w/w aqueous PVP solution was added to the oil concentrate of docetaxel to form a crude emulsion. The resulting emulsion was subjected to high shear dispersion using a Ultraturrax T25 homogenizer.

The resulting coarse emulsion was recycled through Emulsiflex, C5 (Avestin Inc.). The coarse emulsion was re-cycled for 11 passes at 22-25K psi to obtain a fine emulsion. The results of the emulsion formulation stored at 5° C. are shown in Table 1.2.

A study was conducted to evaluate the physical and chemical stability of the docetaxel emulsion. The physical stability (Table 1.2) was evaluated based on the average oil droplet size and percentage of oil droplet population less than 90%. The average oil droplet size was determined using a photon correlation spectroscopy based particle sizing system (Delsa™ Nano analyzer; Beckman Coulter).

TABLE 1.2
Physical Stability of PVP 17 PF Stabilized Docetaxel Emulsion (stored at
5° C.)

| | Initial | 1^{st} Month | 3^{rd} Month | 6^{th} Month |
|---|---|---|---|---|
| Appearance | off-white | off-white | off-white | off-white |
| pH (units) | 5.07 | 5.09 | 5.08 | 5.11 |
| Osmolarity | 290 | 287 | 292 | 281 |
| (mOsm/kg) | | | | |

PSD

| | | | | |
|---|---|---|---|---|
| Average size | 213.2 ± 93.1 | 216.7 ± 82.8 | 212.7 ± 88.8 | 205.7 ± 67.8 |
| (nm) | | | | |
| D (10%) (nm) | 101.3 | 88.1 | 87.4 | 100.8 |
| D (50%) (nm) | 187.5 | 177.3 | 198 | 164.2 |
| D (90%) (nm) | 330.1 | 300.2 | 312.0 | 298.1 |

PSD—particle size distribution;
nm—nanometers;
% w/w—percentage weight/weight;
psi—pounds per square inch

The chemical stability of the docetaxel emulsion was determined based on the concentration of docetaxel in the emulsion over time. The docetaxel concentration in the emulsion was determined by a reverse-phase high pressure chromatography (Waters™ Alliance). The chemical stability of docetaxel emulsion is presented in Table 1.3.

TABLE 1.3
Chemical Stability of PVP 17 PF Stabilized Docetaxel Emulsion

| | Docetaxel Concentration (% w/w) | | | |
|---|---|---|---|---|
| Storage condition | Initial | 1^{st} Month | 3^{rd} Month | 6^{th} Month |
| 5° C. | 95 | 96 | 95 | 95 |
| 25° C./60% RH | | 96 | 94 | 92 |

As evidenced by the results set forth in Tables 1.2 and 1.3, a PVP stabilized docetaxel emulsion showed physical and chemical stability over a 6 month shelf-life period at 5° C.

### Example 2

This example demonstrates the preparation of a stable oil-in-water emulsion of the invention.

A docetaxel emulsion was prepared as described in Table 2.1 using human serum albumin (HSA) as a stabilizer.

TABLE 2.1
Composition of HSA Stabilized Docetaxel Emulsion

| Ingredient | Percentage (% w/w) |
|---|---|
| Docetaxel | 0.5 |
| Soybean oil | 3.1 |
| Miglyol 812N | 3.1 |
| alpha-tocopherol | 0.10 |
| Egg lecithin | 1.0 |
| Cardiolipin | 0.1 |
| HSA | 1.0 |
| Citric acid buffer, pH 5.00 | q.s. 100 |

A solution of 0.5 g/35 ml docetaxel was dissolved in ethanol. Soybean oil, Miglyol 812N and α-tocopherol were added to the ethanol solution with continuous stirring to form an oil phase. Ethanolic solution of egg lecithin and methylene chloride solution of cardiolipin were added to the oil phase. Docetaxel solution was subjected to vacuum evaporation for removal of ethanol. A 1% w/w aqueous solution of HSA was made in citric acid buffer solution. The pH of the citrate buffer was 5.00. Buffer solution was added to the oil concentrate of docetaxel to form a crude emulsion. The emulsion was subjected to high shear dispersion using Ultraturrax T25 homogenizer. The resulting coarse emulsion was recycled through microfluidizer, M-110P (Microfluidics Inc.). The coarse emulsion was re-cycled for 8 passes at 30K psi to obtain a fine emulsion. The results of the emulsion formulation stored at 5° C. are shown in Table 2.2.

A study was conducted to evaluate the physical and chemical stability of docetaxel emulsion. The physical stability (Table 2.2) was evaluated based on the average oil droplet size and percentage of oil droplet population less than 90%. The average oil droplet size was determined using a photon correlation spectroscopy based particle sizing system (Delsa™ Nano analyzer; Beckman Coulter).

TABLE 2.2
Physical Stability of HSA Stabilized Docetaxel Emulsion
(stored at 5° C.)

| | Initial | 1^{st} Month | 3^{rd} Month | 6^{th} Month |
|---|---|---|---|---|
| Appearance | off-white | off-white | off-white | off-white |
| pH (units) | 5.00 | 5.10 | 5.10 | 5.12 |
| Osmolarity | 295 | 298 | 300 | 265 |
| (mOsm/kg) | | | | |

PSD

| | | | | |
|---|---|---|---|---|
| Average size | 273.6 ± 91 | 282 ± 69.6 | 261.8 ± 65.2 | 247.1 ± 44.8 |
| (nm) | | | | |
| D (10%) (nm) | 165.7 | 156.2 | 91.3 | 94.4 |
| D (50%) (nm) | 252 | 248 | 208.0 | 215.3 |
| D (90%) (nm) | 383 | 351.8 | 302.4 | 310.1 |

The chemical stability of docetaxel emulsion was determined based on the concentration of docetaxel in the emulsion over time. The docetaxel concentrations in the emulsion were determined by a reverse-phase high pressure chromatography (Waters™ Alliance). The chemical stability of docetaxel emulsion is presented in Table 2.3.

TABLE 2.3
Chemical Stability of HSA Stabilized Docetaxel Emulsion

| | Docetaxel Concentration (% w/w) | | | |
|---|---|---|---|---|
| Storage condition | Initial | 1^{st} Month | 3^{rd} Month | 6^{th} Month |
| 5° C. | 97 | 96 | 94 | 94 |
| 25° C./60% RH | | 97 | 93 | 94 |

As demonstrated by the results in Examples 1 and 2, both polyvinylpyrrolidone and human serum albumin stabilized emulsions are stable for 6 months at a storage temperature of 5° C. Additionally, the docetaxel concentration and average oil droplet size after a 6 month shelf life are comparable with that of the initial data.

### Example 3

This example demonstrates the preparation of a stable oil-in-water emulsion of the invention.

To demonstrate the stability of high oil content to phospholipid ratio of emulsion, the oil phase was mixed with relatively low lecithin content to obtain the final emulsions containing over 5% oil as shown in Table 3.1.

TABLE 3.1
Composition of High Oil Containing Docetaxel Emulsion

| Ingredient | Percentage (% w/w) |
|---|---|
| Docetaxel | 0.5 |
| Soybean oil | 3.1 |
| Miglyol 812N | 3.1 |
| Egg lecithin | 1.0 |
| Citric acid buffer, pH 5.00 | q.s. 100 |

A solution of 0.5 g/35 mL docetaxel was dissolved in ethanol. Soybean oil and Miglyol 812N were added to the above ethanol solution with continuous stirring to form a oil phase. Ethanolic solution of egg lecithin was added to the oil phase. The docetaxel solution was subjected to vacuum evaporation for removal of ethanol. Citric acid buffer solution, pH 5.00, was added to the oil concentrate of docetaxel to form a crude emulsion. The emulsion was subjected to high shear dispersion using Ultraturrax T25 homogenizer. The resulting coarse emulsion was recycled through microfluidizer, M-110P (Microfluidics Inc.). The coarse emulsion was re-cycled for 8 passes at 30K psi to obtain a fine emulsion.

The physical stability was evaluated based on the average oil droplet size, percentage of oil droplet population less than 90%. The average oil droplet size was determined using a photon correlation spectroscopy based particle sizing system (Delsa™ Nano analyzer; Beckman Coulter). The physical and chemical stability data of the high ratio of oil-to-phospholipid emulsion stored at 5° C. are presented in Tables 3.2 and 3.3, respectively.

TABLE 3.2
Physical Stability of High Oil Containing Docetaxel Emulsion
(stored at 5° C.)

| | Initial | 1^{st} Month | 3^{rd} Month | 6^{th} Month |
|---|---|---|---|---|
| Appearance | off-white | off-white | off-white | off-white |
| pH (units) | 5.04 | 5.03 | 5.01 | 5.05 |
| Osmolarity | 292 | 288 | 292 | 290 |
| (mOsm/kg) | | | | |

PSD

| | | | | |
|---|---|---|---|---|
| Average size | 122.4 ± 55.4 | 163.6 ± 83.1 | 193.6 ± 93.7 | 197.1 ± 63.3 |
| (nm) | | | | |
| D (10%) (nm) | 59.9 | 72.2 | 97.4 | 136.6 |
| D (50%) (nm) | 105.6 | 103.4 | 169.3 | 216.2 |
| D (90%) (nm) | 193.0 | 172.7 | 298.6 | 340.3 |

TABLE 3.3
Chemical Stability of High Oil Containing Docetaxel Emulsion

| | Docetaxel Concentration (% w/w) | | | |
|---|---|---|---|---|
| Storage condition | Initial | 1^{st} Month | 3^{rd} Month | 6^{th} Month |
| 5° C. | 97 | 97 | 96 | 94 |
| 25° C./60% RH | | 97 | 95 | 95 |

Despite the high oil content of the docetaxel emulsion, the docetaxel emulsion showed chemical and physical stability for 6 months at a storage temperature of 5° C.

### Example 4

This example demonstrates the preparation of a stable oil-in-water emulsion of the invention.

To investigate the influence of anti-oxidant, docetaxel emulsions were prepared using alpha-tocopherol and thioglycerol as described in Table 4.1.

TABLE 4.1
Composition of Docetaxel Emulsions Containing Different Anti-oxidants

| Ingredient | Percentage (% w/w) |
|---|---|
| Docetaxel | 0.5 |
| Soybean oil | 3.1 |
| Miglyol 812N | 3.1 |
| Egg lecithin | 1.0 |
| alpha-tocopherol or thioglycerol | 0.5 |
| Citric acid buffer, pH 5.00 | q.s. 100 |

Both the anti-oxidants were used at a concentration of 0.5% w/w in the docetaxel emulsions. A solution of 0.5 g/35 mL docetaxel was dissolved in ethanol. Soybean oil and Miglyol 812N were added to the above ethanol solution with continuous stirring to form a oil phase. Ethanolic solution of egg lecithin was added to the oil phase. During the emulsion preparation, alpha-tocopherol was added to the oil phase, while in the thioglycerol containing emulsion; thioglycerol was added to the aqueous phase/citric acid buffer. In both of the emulsions (alpha-tocopherol or thioglycerol containing emulsions), the docetaxel solution was subjected to vacuum evaporation for removal of ethanol. Citric acid buffer solution, pH 5.00, was added to the oil concentrate of docetaxel to form a crude emulsion. The emulsion was subjected to high shear dispersion using Ultraturrax T25 homogenizer. The resulting coarse emulsion was recycled through microfluidizer, M-110P (Microfluidics Inc.). The coarse emulsion was re-cycled for 8 passes at 30K psi to obtain a fine emulsion.

The physical stability was evaluated based on the average oil droplet size and percentage of oil droplet population less than 90%. The average oil droplet size was determined using a photon correlation spectroscopy based particle sizing system (Delsa™ Nano analyzer; Beckman Coulter). The physical and chemical stability data of alpha-tocopherol and thioglycerol containing docetaxel emulsions are presented in Tables 4.2 and 4.3, respectively.

TABLE 4.2
Physical Stability of Docetaxel Emulsions Containing Different
Anti-oxidants (stored at 5° C.)

| | | | |
|---|---|---|---|
| Initial | 1^{st} Month | 3^{rd} Month | 6^{th} Month |

Alpha-tocopherol Containing Docetaxel Emulsion

| | | | | |
|---|---|---|---|---|
| Appearance | off-white | off-white | off-white | off-white |
| pH (units) | 5.05 | 5.02 | 5.03 | 5.05 |
| Osmolarity | 285 | 280 | 288 | 297 |
| (mOsm/kg) | | | | |
| PSD | | | | |
| Average | 159.5 ± 85.9 | 175.4 ± 91.7 | 196.7 ± 76.2 | 206.7 ± 96.8 |
| size (nm) | | | | |
| D (10%) (nm) | 67.0 | 76.9 | 82.2 | 99.0 |
| D (50%) (nm) | 132.7 | 146.4 | 156.2 | 177 |
| D (90%) (nm) | 269.9 | 292.5 | 301.4 | 329.4 |

Thioglycerol Containing Docetaxel Emulsion

| | | | | |
|---|---|---|---|---|
| Appearance | off-white | off-white | off-white | off-white |
| pH (units) | 5.02 | 5.02 | 5.00 | 5.05 |
| Osmolarity | 285 | 287 | 291 | 277 |
| (mOsm/kg) | | | | |
| PSD | | | | |
| Average | 155.9 ± 64.5 | 170.9 ± 60.2 | 182.2 ± 74.5 | 187.1 ± 65.9 |
| size (nm) | | | | |
| D (10%) (nm) | 80.0 | 97.6 | 94.5 | 106.4 |
| D (50%) (nm) | 137.4 | 153.8 | 160.8 | 168.6 |
| D (90%) (nm) | 237.6 | 245.7 | 275.9 | 268.9 |

TABLE 4.3
Chemical Stability of Docetaxel Emulsions Containing Different Anti-
oxidants (stored at 5° C.)

| Storage | Docetaxel Concentration (% w/w) | | | |
|---|---|---|---|---|
| condition | Initial | 1^{st} Month | 3^{rd} Month | 6^{th} Month |

Alpha-tocopherol Containing Docetaxel Emulsion

| | | | | |
|---|---|---|---|---|
| 5° C. | 93 | 94 | 93 | 92 |
| 25° C./60% RH | | 93 | 91 | 93 |

Thioglycerol Containing Docetaxel Emulsion

| | | | | |
|---|---|---|---|---|
| 5° C. | 94 | 95 | 95 | 95 |
| 25° C./60% RH | | 93 | 94 | 94 |

As demonstrated by the results described above, alpha-tocopherol and thioglycerol containing docetaxel emulsions showed stability over a 6 month shelf-life at 5° C. Both the emulsions showed average oil droplet size of less than 200 nm, and there was no significant difference between the average droplet sizes of docetaxel emulsions over the shelf life period. From the formulation perspective, thioglycerol is easy to solubilize in aqueous phase, and might act as a better anti-oxidant due to its presence in external aqueous phase.

### Example 5

This example demonstrates the preparation of a stable oil-in-water emulsion of the invention.

To investigate the influence of buffer type, docetaxel emulsions were prepared using citric acid, malic acid, succinic acid, tartaric acid and glutaric acid. All of the buffer salts were used at a concentration of 10 mM in the docetaxel emulsions. Docetaxel emulsions with various buffer salts were prepared as described in Table 5.1.

TABLE 5.1
Docetaxel Emulsions Containing Various Buffer Salts at pH 5.00

| Ingredient | Percentage (% w/w) |
|---|---|
| Docetaxel | 0.5 |
| Soybean oil | 3.1 |
| Miglyol 812N | 3.1 |
| Egg lecithin | 1.0 |
| Thioglycerol | 0.5 |
| Citric/malic/succinic/tartaric/glutaric | q.s. 100 |
| acid buffer, pH 5.00 | |

A solution of 0.5 g/35 ml docetaxel was dissolved in ethanol. Soybean oil and Miglyol 812N were added to the above ethanol solution with continuous stirring to form a oil phase. Ethanolic solution of egg lecithin was added to the oil phase. The docetaxel solution was subjected to vacuum evaporation for removal of ethanol. During emulsion preparation, thioglycerol was added to the aqueous/buffer phase. Buffer solution was added to the docetaxel oil concentrate to form a crude emulsion. The crude emulsion was subjected to high shear dispersion using Ultraturrax T25 homogenizer. The resulting coarse emulsion was recycled through microfluidizer, M-110P (Microfluidics Inc.). The coarse emulsion was re-cycled for 8 passes at 30K psi to obtain a fine emulsion.

The physical stability was evaluated based on the average oil droplet size and polydispersity index. The average oil droplet size was determined using a photon correlation spectroscopy based particle sizing system (Delsa™ Nano analyzer; Beckman Coulter). The physical and chemical stability data of docetaxel emulsions stored at 5° C. are presented in Tables 5.2 and 5.3, respectively.

TABLE 5.2
Physical Stability of Docetaxel Emulsions after One Month Storage (at 5° C.)

| | Citric acid | Glutaric acid | Malic acid | Succinic acid | Tartaric acid |
|---|---|---|---|---|---|
| Appearance | off-white | off-white | off-white | off-white | off-white |
| pH (units) | 4.97 | 5.00 | 5.07 | 4.95 | 5.10 |
| Osmolarity | 272 | 291 | 270 | 267 | 284 |
| (mOsm/kg) | | | | | |
| PSD | | | | | |
| Average size | 158.7 ± 62.9 | 161.7 ± 66.4 | 194.4 ± 74.3 | 161.4 ± 39.1 | 191.4 ± 45.4 |
| (nm) | | | | | |

TABLE 5.3
Docetaxel Concentration (5 mg/g) in Various Buffered Emulsions

| | Docetaxel concentration (% w/w) | | | | |
|---|---|---|---|---|---|
| Storage | | Glutaric | | Succinic | Tartaric |
| condition | Citric acid | acid | Malic acid | acid | acid |
| 5° C. | 101 | 100 | 99 | 102 | 101 |
| 25° C./60% RH | 98 | 98 | 97 | 99 | 96 |

All of the buffer salts showed physical and chemical compatibility with docetaxel. The average oil droplet size of docetaxel emulsions stored at 5° C. remained less than 200 nm. No degradation impurities were observed in docetaxel HPLC analysis.

### Example 6

This example demonstrates the preparation of a stable oil-in-water emulsion of the invention.

The effect of microfluidization passes on the average oil droplet size of the docetaxel emulsion was investigated using the formula set forth in Table 6.1.

TABLE 6.1
Docetaxel Emulsion Composition for Investigation of Microfluidization
Passes

| Ingredient | Percentage (% w/w) |
|---|---|
| Docetaxel | 0.5 |
| Soybean oil | 3.1 |
| Miglyol 812N | 3.1 |
| Egg lecithin | 5.1 |
| Thioglycerol | 0.5 |
| Citric acid buffer, pH 5.00 | q.s. 100 |

A solution of 0.5 g/35 ml docetaxel was dissolved in ethanol. Soybean oil and Miglyol 812N were added to the above ethanol solution with continuous stirring to form a oil phase. Ethanolic solution of egg lecithin was added to the oil phase. The docetaxel solution was subjected to vacuum evaporation for removal of ethanol. Thioglycerol was added to citric acid buffer, pH 5.00, which in turn was added to the oil concentrate of docetaxel to form a crude emulsion. The emulsion was subjected to high shear dispersion using Ultraturrax T25 homogenizer. The resulting coarse emulsion was re-cycled through microfluidizer, M-110P (Microfluidics Inc.). The coarse emulsion was re-cycled for different passes (e.g., 4, 6, 8 and 12) at 30K psi to obtain a fine emulsion.

The particle size distribution was evaluated based on the average oil droplet size and polydispersity index. The average oil droplet size was determined using a photon correlation spectroscopy based particle sizing system (Delsa™ Nano analyzer; Beckman Coulter). The physical and chemical stability data of docetaxel emulsions stored at 5° C. are presented in Tables 6.2 and 6.3, respectively.

TABLE 6.2
Physical Stability of Docetaxel Emulsion at Different Microfluidization
Passes

| | Pass #4 | Pass #6 | Pass #8 | Pass #12 |
|---|---|---|---|---|
| Appearance | Slight | off-white | off-white | off-white |
| | yellowish | | | |
| pH (units) | 5.01 | 5.02 | 5.00 | 5.01 |
| Osmolarity | 294 | 293 | 287 | 286 |
| (mOsm/kg) | | | | |
| Average | 137.8 ± 55.9 | 124.6 ± 36.8 | 110.1 ± 50.5 | 103.4 ± 38.7 |
| size (nm) | | | | |

TABLE 6.3
Chemical Stability of Docetaxel Emulsion at Different Microfluidization
Passes

| | Pass #4 | Pass #6 | Pass #8 | Pass #12 |
|---|---|---|---|---|
| Initial Conc. | 5.10 | 4.90 | 4.90 | 4.89 |
| (mg/mL) | | | | |

As demonstrated by the above-described results, oil droplets showed a slight reduction in particle size with the number of passes. At the 8^{th} pass, further reduction in droplet size ceased. No significant changes were observed in osmolarity of the docetaxel emulsions with pass number. After the 6^{th} pass, docetaxel concentration remained the same. From the data, it is inferred that 6 to 8 passes would be optimum to obtain the average oil droplet size in the range of 100-120 nm.

### Example 7

This example demonstrates the bioavailability of a stable oil-in-water emulsion of the invention.

In order to test the bio-equivalency, the inventive docetaxel emulsion was screened against the commercial TAXOTERE™ formulation. TAXOTERE™ formulation (Sanofi-Aventis) is polysorbate concentrate of docetaxel available in concentrations of 20 mg/0.5 mL, 40 mg/mL and 80 mg/2 mL.

The docetaxel emulsion is a lipid based formulation at a concentration of 5 mg/mL. The docetaxel emulsion was prepared as described in Example 6. The resulting emulsion was filtered into glass vials through 0.22 μm filter. The initial average particle size of the emulsion droplets was measured to be 97.2±44.5 nm. The docetaxel concentration in the emulsion was determined to be 94% w/w by reverse-phase high pressure chromatography. The zeta potential of the emulsion droplets was determined to be −47 mV using Delsa™ Nano analyzer (Beckman Coulter). The physical stability results of the emulsion formulation stored at 5° C. are shown in Table 7.1.

TABLE 7.1
Physical Stability of Docetaxel Emulsion used in Rat Pharmacokinetic
Studies (stored at 5° C.)

| | Initial | 1^{st} Month | 3^{rd} Month | 6^{th} Month |
|---|---|---|---|---|
| Appearance | off-white | off-white | off-white | off-white |
| pH (units) | 5.02 | 5.03 | 5.02 | 5.12 |
| Osmolarity | 290 | 286 | 280 | 279 |
| (mOsm/kg) | | | | |
| PSD | | | | |
| Average | 97.2 ± 44.5 | 148.5 ± 58.2 | 134.2 ± 31.1 | 141.2 ± 49.5 |
| size (nm) | | | | |
| D (10%) (nm) | 46.1 | 79.1 | 92.7 | 80.5 |
| D (50%) (nm) | 84.2 | 132.0 | 124.9 | 127.0 |
| D (90%) (nm) | 154.2 | 221.8 | 171.0 | 203.4 |

Studies were conducted to evaluate the chemical stability of docetaxel. The chemical stability was determined based on concentration of docetaxel in the emulsion over time. The docetaxel concentration was determined by a reverse-phase high pressure chromatography. The chemical stability of docetaxel is presented in Table 7.2.

TABLE 7.2
Chemical Stability of Docetaxel Emulsion (5 mg/g Docetaxel
Concentration) used in Rat Pharmacokinetic Studies

| Storage | Docetaxel Concentration (% w/w) | | | |
|---|---|---|---|---|
| condition | Initial | 1^{st} Month | 3^{rd} Month | 6^{th} Month |
| 5° C. | 94 | 94 | 95 | 94 |
| 25° C./60% RH | | 94 | 94 | 93 |
| 40° C./75% RH | | 93 | 93 | 93 |

The docetaxel emulsion at 5 mg/mL concentration showed physical and chemical stability. Average oil droplet size of docetaxel emulsions stored at 5° C. remained less than 200 nm.

An intravenous bolus injection of docetaxel emulsion and TAXOTERE™ was administered in Wistar rats (Rattus Norvegicus). Post drug administration, blood samples were collected at different time points and evaluated for docetaxel concentration in plasma. The data was subjected to WinNolin analysis to derive pharmacokinetic parameters.

The animals were observed for any unusual clinical signs until 30 minutes post-injection. All animals were found healthy with no significant behavioral changes during the course of experiment. Both formulations were administered at a dose of 2.5 mg/kg using a sterile 1 mL disposable syringe and a 26G needle. Blood samples (approximately 150 μL) were drawn from the animals by retro-orbital bleeding under mild ether anesthesia at time points of 0 h (prior to dosing), 0.08 h, 0.17 h, 0.33 h, 0.50 h, 1.0 h, 2.0 h, 4.0 h, 8.0 h and 24.0 h post administration into silicon coated tubes containing EDTA. Plasma was separated from these samples immediately by centrifuging at 4000 rpm for 5 minutes. The separated plasma from the samples was transferred to eppendorf tubes and stored immediately at −80° C. until analysis by LC-MS/MS.

Frozen plasma samples obtained from the study were thawed at room temperature and used for further processing. For analysis, a 50 μL plasma sample was taken. The samples were analyzed using TBME (tert-butyl-methyl ether) as extraction solvent and LC-MS/MS. Pharmacokinetic parameters were evaluated from the concentration profile of the test items using WinNolin software (version 5.0.1). The pharmacokinetic parameters of TAXOTERE™ and docetaxel emulsion are shown in Table 7.3.

TABLE 7.3
Comparative PK Parameters of TAXOTERE ™ and Docetaxel Emulsion
in Rats

| | | Docetaxel | TAXOTERE ™ ± |
|---|---|---|---|
| Parameter | Units | Emulsion ± SD | SD |
| HL_Lambda_z | hr | 0.824 ± 0.3025 | 1.31 ± 0.70 |
| C0 | {ng/ | 1708.05 ± 482.48 | 910.76 ± 266.9 |
| | ml} | | |
| AUClast | hr * | 152.95 ± 32.092 | 223.42 ± 82.27 |
| | {ng/ | | |
| | ml} | | |
| AUCINF_obs | hr * | 169.928 ± 34.774 | 248.50 ± 88.51 |
| | {ng/ | | |
| | ml} | | |
| Vz_obs | ml/kg | 17448.95 ± 4382.70 | 18457.80 ± 7780.75 |
| Cl_obs | ml/ | 14791.815 ± 3588.1191 | 10988.64 ± 3270.14 |
| | hr/kg | | |

Pharmacokinetic profiles of the two formulations were compared. The Cₘₐₓ value of docetaxel in the docetaxel emulsion and TAXOTERE™ were comparable. No significant difference was observed in the area under curve (AUC) of docetaxel in docetaxel emulsion (152.95±32.092 hr*ng/ml) and TAXOTERE™ (223.42±82.27 hr*ng/ml). However, clearance was found to be higher in the docetaxel formulation (14791.815±3588.1191 ml/hr/kg) as compared to TAXOTERE™ (10988.64±3270.14).

In summary, both TAXOTERE™ and the docetaxel emulsion were found to show comparable bioavailability with respect to Cₘₐₓ and AUC (last).

### Example 8

This example demonstrates the preparation of a stable oil-in-water emulsion of the invention.

A lecithin stabilized emulsion was prepared using a different mode of addition of ingredients. The method and composition of the prepared emulsion is described in Table 8.1.

TABLE 8.1
Docetaxel Emulsion Composition

| Ingredient | Percentage (% w/w) |
|---|---|
| Docetaxel | 0.5 |
| Soybean oil | 3.1 |
| Miglyol 812N | 3.1 |
| Egg lecithin | 5.1 |
| Thioglycerol | 0.5 |
| Citric acid buffer, pH 5.00 | q.s. 100 |

A solution of 0.5 g/35 ml docetaxel was dissolved in ethanol. Soybean oil and Miglyol 812N were added to the docetaxel solution. The docetaxel solution was subjected to vacuum evaporation for removal of ethanol. Thioglycerol was added to citric acid buffer. Egg lecithin was dispersed in citrate buffer. The pH of the lecithin dispersed buffer was adjusted to 5.00. Lecithin dispersion was added to docetaxel concentrate with continuous stirring to form a crude emulsion. The emulsion was subjected to high shear dispersion using Ultraturrax T25 homogenizer (20,000 RPM at room temperature for 5 minutes). The resulting coarse emulsion was re-cycled through microfluidizer, M-110P (Microfluidics Inc.). The coarse emulsion was re-cycled for 8 passes at 30K psi to obtain a fine emulsion.

The physical stability data of the emulsion formulation stored at 5° C. is shown in Table 8.2.

TABLE 8.2
Physical Stability of Docetaxel Emulsion

| | Initial | 1^{st} Month | 3^{rd} Month | 6^{th} Month |
|---|---|---|---|---|
| Appearance | off-white | off-white | off-white | off-white |
| pH (units) | 5.12 | 5.08 | 5.09 | 5.07 |
| Osmolarity | 291 | 295 | 292 | 277 |
| (mOsm/kg) | | | | |
| PSD | | | | |
| Average size | 110.1 ± 50.5 | 106.2 ± 43.3 | 117.4 ± 38.4 | 127.9 ± 41.2 |
| (nm) | | | | |
| D (10%) (nm) | 64.5 | 55.2 | 65.7 | 67.4 |
| D (50%) (nm) | 94.6 | 93.7 | 97.8 | 101.2 |
| D (90%) (nm) | 138.5 | 161.3 | 171.0 | 170.8 |

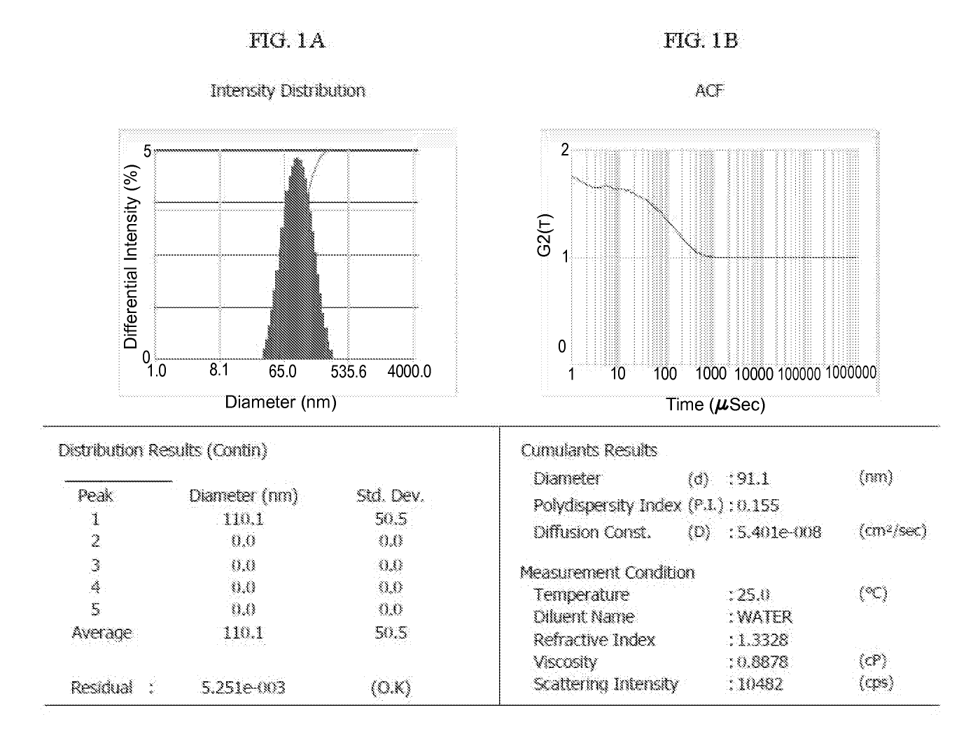

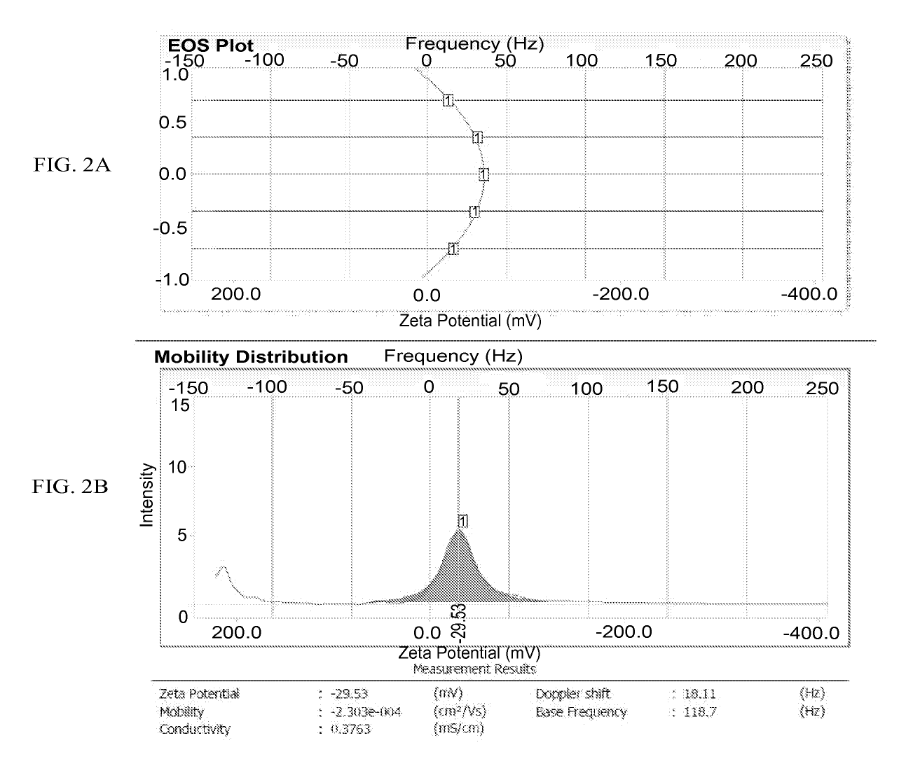

The initial average particle size and zeta potential of the emulsion droplets are shown in FIGS. 1 and 2. The docetaxel concentration in the emulsion was determined to be 96% w/w by reverse-phase high pressure chromatography method.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pharmaceutical composition comprising:
(a) a taxane;
(b) an oil phase, wherein the oil phase is present in an amount at least about 4.0% w/w of the total composition;
(c) an emulsifier, wherein the emulsifier is present in an amount less than about 1.2% or more than 5.0% w/w of the total composition; and
(d) an aqueous phase.

2. The pharmaceutical composition of claim 1, wherein the oil phase comprises vegetable oil, medium chain triglycerides, or mixtures thereof.

3. The pharmaceutical composition of claim 2, wherein the vegetable oil is a refined soybean oil or safflower oil.

4. The pharmaceutical composition of claim 2, wherein the medium chain triglycerides have an aliphatic carbon chain length between C8 to C12.

5. The pharmaceutical composition of claim 4, wherein the medium chain triglycerides are selected from a group consisting of caproic, caprylic, capric and lauric triglycerides, and mixtures thereof.

6. The pharmaceutical composition of claim 2, wherein the vegetable oil is present in an amount of at least about 3.0% w/w of the total composition, and the medium chain triglycerides are present in an amount of at least about 3.0% w/w of the total composition.

7. The pharmaceutical composition of claim 1, wherein the emulsifier comprises a phospholipid.

8. The pharmaceutical composition of claim 7, wherein the emulsifier is egg lecithin.

9. The pharmaceutical composition of claim 1, wherein the emulsifier comprises at least two phospholipids.

10. The pharmaceutical composition of claim 9, wherein at least one of the phospholipids is selected from the group consisting of lecithin, phosphatidylcholine, phosphatidylethanolamine, phosphatidylserine, phosphatidylinositol, phosphatidic acid, alkali metal salts thereof, quaternary ammonium salts thereof, and mixtures thereof.

11. The pharmaceutical composition of claim 9, wherein at least one of the phospholipids is cardiolipin.

12. The pharmaceutical composition of claim 9, wherein the phospholipids are egg lecithin and cardiolipin.

13. The pharmaceutical composition of claim 7, wherein the phospholipid is present in an amount of less than about 1.2% w/w of the total composition.

14. The pharmaceutical composition of claim 7, wherein the phospholipid is present in an amount of more than 5.0% w/w of the total composition.

15. The pharmaceutical composition of claim 1, wherein the composition further comprises a polyvinylpyrrolidone homopolymer or an emulsion stabilizing protein.

16. The pharmaceutical composition of claim 15, wherein the polyvinylpyrrolidone homopolymer is polyvinylpyrrolidone present in an amount of about 0.05% to about 50.0% w/w of the total composition.

17. The pharmaceutical composition of claim 15, wherein the emulsion stabilizing protein is human serum albumin present in an amount of about 0.05% to about 50.0% w/w of the total composition.

18. The pharmaceutical composition of claim 1, wherein the taxane is docetaxel.

19. The pharmaceutical composition of claim 18, wherein the docetaxel is present in an amount of about 0.01% to about 10.0% w/w of the total composition.

20. The pharmaceutical composition of claim 1, wherein the composition further comprises a buffer made from the alkali salts of citric acid, acetic acid, maleic acid, phosphoric acid, succinic acid, or tartaric acid.

21. The pharmaceutical composition of claim 20, wherein the composition has a pH of about 3 to about 7.

22. The pharmaceutical composition of claim 1, wherein the composition further comprises an anti-oxidant selected from the group consisting of butylated hydroxytoluene, butylated hydroxytoluene, a-tocopherol, sodium ascorbate, thioglycerol, and ethylene di-amine tetra acetic acid, wherein the anti-oxidant is present in an amount between 0.005% and 5% w/w of the total composition.

23. The pharmaceutical composition of claim 1, wherein the composition further comprises a lyoprotective agent, an isotonicity adjusting agent, or combinations thereof.

24. The pharmaceutical composition of claim 23, wherein the lyoprotective agent is selected from the group consisting of sugars, polymers, and amino acids.

25. The pharmaceutical composition of claim 18, wherein the composition is in the form of an oil-in-water emulsion.

26. The pharmaceutical composition of claim 25, wherein the pharmaceutical composition is diluted or reconstituted using intravenous diluents to obtain an intravenous infusion which is ready for administration.

27. The pharmaceutical composition of claim 26, wherein the total volume of infusion of each effective dose to be administered to a patient is less than 300 ml of docetaxel containing dispersion comprising solid particles or liquid droplets, wherein the average diameter of the particles or droplets is less than 1 micro-meter.

28. A pharmaceutical composition comprising:
(a) docetaxel;
(b) an oil phase comprising soybean oil and medium chain triglycerides, wherein the oil phase is present in an amount of at least about 4.0% w/w of the total composition, wherein the soybean oil is present in an amount of at least about 3.0% w/w of the total composition, and wherein the medium chain triglycerides are present in an amount of about 3.0% w/w of the total composition;
(c) an emulsifier selected from the group consisting of phospholipids, cholesterol, cholesterol derivatives, cholesterol salts, cardiolipin, cardiolipin derivatives, and mixtures thereof, wherein the emulsifier is present in an amount less than about 1.2% w/w or more than 5.0% w/w of the total composition;
(d) an aqueous phase selected from the group consisting of an aqueous solution of polyvinylpyrrolidone, an aqueous solution of human serum albumin, and combinations thereof, wherein the aqueous phase is present in an amount of about 0.05% to about 50.0% w/w of the total composition.

29. An oil-in-water emulsion comprising:
(a) docetaxel in an amount of about 0.1% to about 10.0% w/w of the total emulsion;
(b) an oil phase comprising soybean oil, medium chain triglycerides, and alpha-tocopherol, wherein the oil phase is present in an amount of at least about 4.0% w/w of the total emulsion, wherein the soybean oil is present in an amount of at least about 3.0% w/w of the total emulsion, and wherein the medium chain triglycerides are present in an amount of about 3.0% w/w of the total emulsion;
(c) an emulsifier comprising egg lecithin and cardiolipin, wherein the emulsifier is present in an amount less than about 1.2% w/w of the total composition;
(d) an aqueous phase selected from the group consisting of an aqueous solution of polyvinylpyrrolidone, an aqueous solution of human serum albumin, and mixtures thereof, wherein the aqueous phase is present in an amount of about 0.05% to about 50.0% w/w of the total emulsion.

30. A method of treating cancer in a human patient comprising administering a therapeutically effective amount of the pharmaceutical composition of claim 1.

31. A method of administering a pharmaceutical composition of claim 1, wherein the pharmaceutical composition of docetaxel is administered by intravenous route to patients suffering from cancer.

32. A method of administering a pharmaceutical composition of claim 1, wherein the pharmaceutical composition of docetaxel is administered by intramuscular or subcutaneous or intra-arterial or oral routes for treatment of patients suffering from cancer.
